(19) United States
(12) Patent Application Publication
(10) Pub. No.: US 2005/0276780 A1
(43) Pub. Date: Dec. 15, 2005

(54) Shaving composition

(76) Inventor: William A. King, Buckinghamshire (GB)

Correspondence Address: DICKSTEIN SHAPIRO MORIN & OSHINSKY LLP, 2101 L Street NW, Washington, DC, 20037-1526, US

(21) Appl. No.: 11/138,359

(22) Filed: May 27, 2005

(30) Foreign Application Priority Data
May 27, 2004 (GB) 0411887.3

(51) Int. Cl. A61K 7/15

(52) U.S. Cl. 424/73

(57) ABSTRACT

The shaving composition, in particular a gel, is magnetic. The composition contains ferromagnetic particles, in particular magnetic iron oxide.

### BACKGROUND OF THE INVENTION

1. Field of Invention

This invention relates to shaving compositions, which are applied to the skin to facilitate shaving.

2. Background Art

Shaving compositions have been available for decades in a variety of forms including bars, sticks, creams, lotions, and foams (both aerosol or non-aerosol). Such compositions or gels provide a form of lubrication to the shaving surface prior to shaving, to reduce friction, and may also offer a level of skin protection, nourishment, healing, and moisturization.

After shaving, the razor is usually rinsed, in order to wash the conventional shaving composition off the blade or blades.

### SUMMARY OF INVENTION

In one aspect invention provides a shaving composition which is magnetic.

In another aspect the invention provides a shaving composition containing ferromagnetic particles.

In another aspect the invention provides a shaving composition containing magnetic iron oxide or magnetite (Fe₃O₄).

When applied to the shaving surface, the razor will come in contact with the shaving composition and a proportion of the composition will be retained on the razor blade(s) due to the magnetic nature of the blade(s). The resulting coating of shaving composition on the surface of the blade(s) serves as a thin film coating which is retained until the razor is next used, protecting the blade(s) from the primary cause of deterioration, being oxidation, and ensuring that an additional level of lubrication is immediately available already on the blade ready for its next use.

It thus becomes possible to provide a shaving composition or gel which not only protects, nourishes, and moisturizes the skin being shaved (shaving surface) but also protects the razor and reduces the rate of deterioration and erosion due to the shaving action and the atmosphere.

### DETAILED DESCRIPTION OF THE INVENTION

The shaving composition may be based on any conventional shaving composition (whether liquid, flowable, or spreadable), with the addition of a suitable amount of ferromagnetic particles.

Any suitable ferromagnetic particles may be used, for example iron oxide or chromium dioxide.

The minimum content of ferromagnetic particles is determined by the need to provide a significant magnetic effect and may, for example, be at least about 0.01 wt. %, preferably at least about 0.02 wt. %, more preferably at least about 0.03 wt. %. The maximum content is limited by the desired physical properties of the composition, such as viscosity, translucence, and color. For example, the content of ferromagnetic particles may be at most about 1 wt. %, preferably at most about 0.5 wt. %, more preferably at most about 0.1 wt. %. The content is preferably kept low in order to avoid discoloration of the composition, particularly by iron oxide.

The composition is preferably in the form of a colorless translucent or transparent gel.

Suitable compositions are water-based compositions containing oils and surfactants.

The composition is preferably neutral.

### EXAMPLE 1

### Gel

| | wt. % |
|---|---|
| Water (Aqua) | balance |
| Glycerin | 1-5 |
| Sodium laureth sulfate 28% & Benzyl alcohol (0.02%) | 15-25 |
| & Methylchloroisothiazolinone(0.0005%) & | |
| Methylisothiazolinone (0.0002) | |
| Aloe Barbadensis leaf juice | 5-15 |
| Acrylates/C10-30 alkyl acrylate crosspolymer | 1-3 |
| Triethanolamine | 0.5-1.5 |
| Cocamidopropyl betaine 36% | 0.2-1.0 |
| Octacare F100 ™ | 0.2-0.8 |
| Water (Aqua) 84.533% & Propylene glycol 8.3% & | 0.1-0.8 |
| Vitis Vinifera (Grape) seed oil 3.3% & Mica 1.4% & | |
| Tocopheryl acetate 0.167% & Polyquaternium-11 1% | |
| & Agar 1% & Titanium dioxide (CI 77891) 0.3% | |
| Polypore ™ Grapseed/vitamin (E) | 0.1-0.5 |
| Melaleuca Alternifolia (Tea Tree) leaf oil | 0.1-0.5 |
| Eucalyptus Globulus leaf oil | 0.1-0.5 |
| Methyldibromo glutaronitrile 20% & Phenoxyethanol | 0.05-0.2 |
| 80% | |
| Benzophenone-4 | 0.05-0.2 |
| Magnetite | 0.01-0.5 |
| Mentha Viridis (Spearmint) leaf oil | 0.01-0.1 |
| Camphor | 0.01-0.1 |
| Hydrochloric acid | 0.01-0.05 |
| Ext Violet 2 (CI 60730) | 0.0001-0.001 |
| Blue 1 (CI 42090) | 0.0001-0.001 |

### EXAMPLE 2

### Oil

| | wt. % |
|---|---|
| Vitis Vinifera (Grape) Seed Oil | balance |
| Aloe Barbadensis leaf juice | 0.5-2% |
| MAGNETITE | 0.01-0.5% |
| PEG 40 Hydrogenated Castor Oil | 0.2-1.0% |
| Eucalyptus globulus leaf oil | 0.2-1.0% |
| Tocopheryl Acetate (vitamin E acetate) | 0.1-0.5% |
| Melaleuca alternifolia (Tea tree) oil | 0.05-0.2% |
| Mentha viridis (Spearmint) leaf oil | 0.01-0.1% |
| Cinnamomum Camphora (Camphor) | 0.01-0.05% |
| Bark Oil | |
| CI 61565 (Green 6) | 0.01-0.05% |

1. A shaving composition which is magnetic.

2. A shaving composition containing ferromagnetic particles.

3. A shaving composition as claimed in claim 2, in which the content of ferromagnetic particles is at least about 0.01 wt. %.

4. A shaving composition as claimed in claim 3, in which said content is at least about 0.02 wt. %.

5. A shaving composition as claimed in claim 3, in which said content is at least about 0.03 wt. %.

6. A shaving composition as claimed in claim 3, in which the content of ferromagnetic particles is at most about 1 wt. %.

7. A shaving composition as claimed in claim 6, in which said content is at most about 0.5 wt. %.

8. A shaving composition as claimed in claim 6, in which said content is at most about 0.1 wt. %.

9. A shaving composition containing magnetic iron oxide.

10. A shaving composition as claimed in claim 9, in which the content of iron oxide is at least about 0.01 wt. %.

11. A shaving composition as claimed in claim 10, in which the content of iron oxide is at most about 1 wt. %.

12. A shaving composition as claimed in claim 1, in the form of a gel.

13. A shaving composition as claimed in claim 1, in which the shaving composition is colorless.

14. A shaving composition as claimed in claim 1, in which the shaving composition is translucent or transparent.

15. A shaving composition as claimed in claim 2, in the form of a gel.

16. A shaving composition as claimed in claim 9, in the form of a gel.

17. A shaving composition as claimed in claim 2, in which the shaving composition is colorless.

18. A shaving composition as claimed in claim 9, in which the shaving composition is colorless.

19. A shaving composition as claimed in claim 2, in which the shaving composition is translucent or transparent.

20. A shaving composition as claimed in claim 9, in which the shaving composition is translucent or transparent.
